(19) United States
(12) Patent Application Publication
(10) Pub. No.: US 2016/0216283 A1
(43) Pub. Date: Jul. 28, 2016

(54) SYSTEMS, COMPOSITIONS, AND METHODS OF LIPID PANEL TEST CONTROLS UTILIZING PARTICLES THAT MIMIC HEMATOCRIT

(71) Applicant: Polymer Technology Systems, Inc., Indianapolis, IN (US)

(72) Inventors: Gary L. Hughes, Anderson, IN (US); Keith Moskowitz, Indianapolis, IN (US); Aniruddha Patwardhan, Fishers, IN (US)

(21) Appl. No.: 15/004,589

(22) Filed: Jan. 22, 2016

Related U.S. Application Data
(60) Provisional application No. 62/107,851, filed on Jan. 26, 2015.

(51) Int. Cl. G01N 33/96

(52) U.S. Cl. CPC G01N 33/96; G01N 2496/10

(57) ABSTRACT

A calibration sample includes serum/plasma having a known level of a first analyte and a plurality of particles that mimic the characteristics of red blood cells.

### CROSS-REFERENCE TO RELATED APPLICATIONS

This Application claims the benefit of Provisional Application No. 62/107,851 filed on Jan. 26, 2015, titled “Systems, Compositions, And Methods Of Lipid Panel Test Controls Utilizing Particles That Mimic Hematocrit,” the entire disclosure of which is hereby incorporated by reference.

### BACKGROUND

Vertical flow test strips are a unique and innovative medium providing for point-of-care testing of blood analytes. While this technology can approach the accuracy provided by laboratory techniques, there are many peculiarities related to these test strips that make their operation, construction, use, and calibration difficult.

One such issue relates to the provision of standards for calibrating the test strips. Many commercial entities provide plasma or serum solutions that can be used as standards for calibrating test strips and other analyte measurement devices. Plasma and serum, however, exclude many parts of whole blood that may affect performance in vertical flow test strips. Therefore, they may not function properly in calibration functions.

### BRIEF SUMMARY

In one embodiment, a calibration sample includes serum/plasma having a known level of a first analyte; and a plurality of particles that mimic the characteristics of red blood cells. In one alternative, the plurality of particles is of red florescent particles. In another alternative, the red florescent particles are polystyrene. Optionally, the red florescent particles are selected from the group consisting of other polymeric material and silica based particles. Alternatively, an amount of red florescent particles added to the serum/plasma is 20% of the plasma volume. In one configuration, the amount of red florescent particles added to the serum/plasma is between 5% and 50% of the plasma volume. In another configuration, the plurality of particles is non-dyed particles. Optionally, an amount of non-dyed particles added to the serum/plasma is 20% of the plasma volume. Alternatively, the amount of non-dyed particles added to the serum/plasma is between 5% and 50% of the plasma volume. Optionally, the first analyte is one of a plurality of analytes, the plurality of analytes consisting of HDL, LDL, triglycerides, creatinine, ketone, total cholesterol, and glucose.

In one embodiment, a method of improving a calibration sample for use with a vertical flow test strip includes providing serum/plasma having a known level of a first analyte; and adding a plurality of particles that mimic the characteristics of red blood cells. Alternatively, the plurality of particles is of red florescent particles. Optionally, an amount of red florescent particles added to the serum/plasma is 20% of the plasma volume. In one alternative, the amount of red florescent particles added to the serum/plasma is between 5% and 50% of the plasma volume.

In one embodiment, a method of calibrating a meter, the meter utilizing a vertical flow test strip, includes providing serum/plasma having a known level of a first analyte, and adjusting the viscosity of the serum/plasma by adding a plurality of particles that mimic the characteristics of red blood cells.

In one embodiment, a method of adjusting calibration results of a vertical flow test strip having a limited dynamic range, calibrated with a calibration sample, the calibration sample including serum/plasma having a known level of a first analyte, includes adding a plurality of particles that mimic the characteristics of red blood cells. Optionally, the plurality of particles is of red florescent particles. Alternatively, an amount of red florescent particles added to the serum/plasma is 20% of the plasma volume. In one alternative, the amount of red florescent particles added to the serum/plasma is between 5% and 50% of the plasma volume. Optionally, the first analyte is HDL. Alternatively, the calibration results are adjusted down to be within the upper limit of the limited dynamic range.

### BRIEF DESCRIPTION OF THE FIGURES

FIG. 1 shows a graph of the non-overlap of the dynamic range for cholesterol and HDL and other analytes in a vertical flow test strip;

FIGS. 2 and 3 show synergent controls analyses for a lipid panel test strip as compared to a reference analyzer;

FIG. 4 shows an image of diluted whole blood red blood cells;

FIG. 5 shows an image of Red Florescent Particles (RFPs);

FIG. 6 shows an image of RFPs trapped in a D-23 glass fiber blood separation layer; and

FIG. 7 shows a graph of how RFPs enable HDL control reading.

### DETAILED DESCRIPTION

Certain terminology is used herein for convenience only and is not to be taken as a limitation on the embodiments of the systems, compositions, and methods of lipid panel test controls utilizing particles that mimic hematocrit. In the drawings, the same reference letters are employed for designating the same elements throughout the several figures.

As noted above, many commercial solutions for calibration of blood analyte detection devices have disadvantages for calibrating vertical flow test strips. Serum and plasma do not have all parts included in whole blood and, therefore, perform differently when applied to vertical flow test strips. These commercial solutions may perform well on central analyzers but, due to a variety of factors, include formulation differences among test strips and reaction kinetics/viscosity of blood concerns, as well as having issues in vertical flow test strips, especially in relation to HDL.

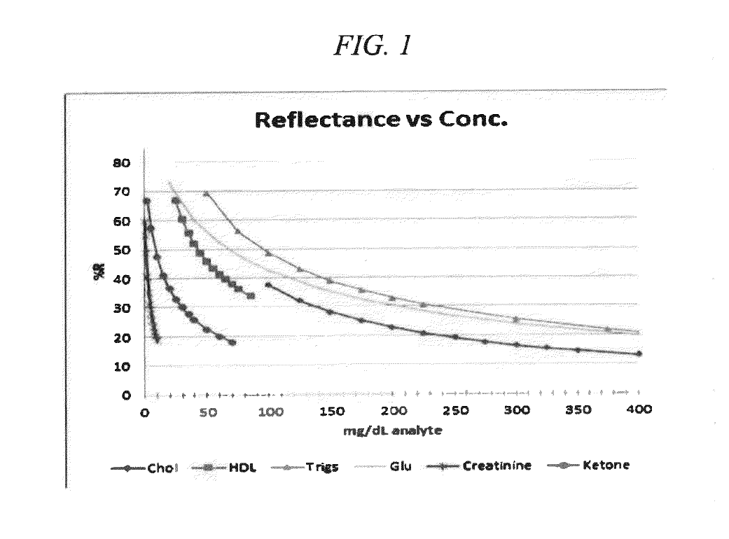

FIG. 1 shows a graph of the non-overlap of the dynamic range for cholesterol and HDL and other analytes in a vertical flow test strip. In FIG. 1, the x-axis relates to the mg/dL of analytes, and the y-axis relates to the reflectance % R.

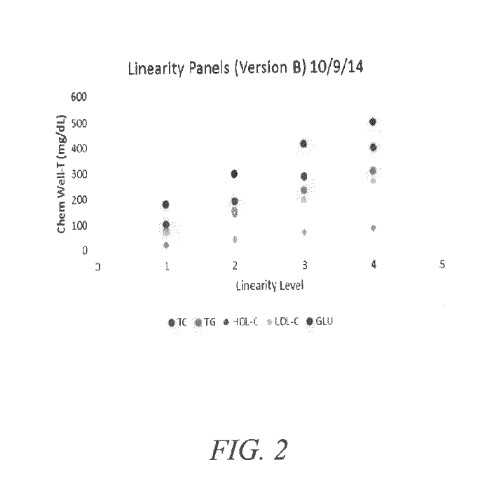

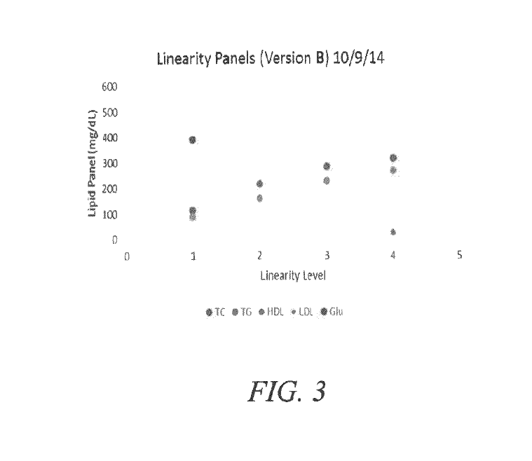

FIGS. 2 and 3 show Lipid controls analyses for a lipid panel test strip as compared to a reference analyzer. Linearity Panels generally are used to calibrate a meter and test strip combination and determine the accuracy and precision of the device. FIG. 2 shows a reference analyzer calibrated using commercial Linearity Panels. FIG. 2 shows relatively good precision and accuracy. In contrast, in tests using the CardioChek Plus®, which is a meter and vertical flow test strip system, there are a number of issues with the accuracy and precision. First, the value for glucose at linearity level 1 is off the scale. This is a significant accuracy issue. The same holds true for HDL at linearity level 4. Finally, the total cholesterol and the HDL appear to plateau or otherwise approach an asymptote at higher linearity levels. More closely mirroring the viscosity and kinetics of whole blood is thought to improve performance in vertical flow test strips. FIG. 3 may provide what appear to be inconsistent results, due to the over-recovery show; however, this is due to electrochemically active components in the control matrix. Additionally, as shown, the plateauing of HDL and cholesterol may be related to the content of the control solution, as opposed to the validity of the testing itself.

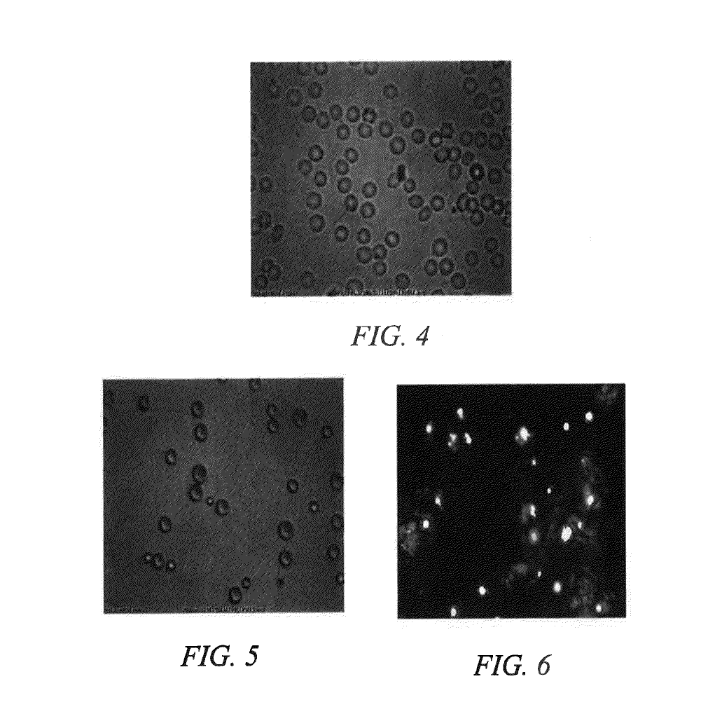

To address this issue, in one embodiment, Red Florescent Particles (RFPs) are utilized. In alternatives, RFPs may include any type of small particle that affects the viscosity in a sample such that the RFPs mimic red blood cells. In many embodiments, the RFPs are composed of Polystyrene. FIG. 4 shows an image of diluted whole blood red blood cells. Red blood cells have an approximate radius of 6 to 8 micrometers. FIG. 5 shows an image of Red Florescent Particles (RFPs). RFPs have a radius of approximately 7 micrometers. The RFPs become stuck in glass matrixes or other matrixes typically in vertical flow test strips. FIG. 6 shows an image of RFPs trapped in a D-23 glass fiber blood separation layer. Such a blood separation layer is commonly used in vertical flow test strips.

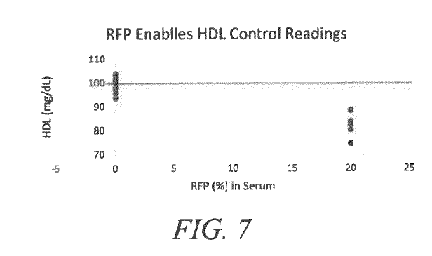

In one embodiment, a percentage of RFPs is added to a control solution in order to normalize performance in calibration of a vertical flow test strip. It is believed that the RFPs affect the viscosity and other performance of the control solutions in a manner mimicking the conditions that exist in whole blood. FIG. 7 shows a graph of how RFPs enable HDL control readings. In the graph, the y-axis represents HDL in mg/dL, and the x-axis represents the RFP percentage added to Serum. One limitation of the dynamic range for HDL is that the maximum is approximately 100 mg/dL. This can be observed in FIG. 1. Therefore, measurements of greater than 100 are off the scale. As shown in FIG. 7 and Table 1 below, with 20% RFPs added to Serum used for calibration, none of the readings for HDL are off the scale. Additionally, p<0.001 t-test (mean) and P<0.05 Fisher's (#<100). According to the p values, the hypothesis is for using 20% RFP to improve HDL results, and the hypothesis should be accepted as true.

TABLE 1

| Data analyzed | 0% RFPs | 20% RFPs | Total |
|---|---|---|---|
| Off Scale (>100) | 5 | 0 | 5 |
| Reading | 5 | 10 | 15 |
| Total | 10 | 10 | 20 |

Therefore, adding RFPs to calibration serum or plasma serum may be a viable technique for improving calibration serum or plasma serum that is used with vertical flow test strips. The RFPs proposed in many places herein are described as florescent polystyrene beads. In alternatives, carboxylate-modified polystyrene latex beads may be substituted. In alternatives, amine-modified polystyrene latex beads may be substituted. In alternatives, other beads may be substituted that are non-reactive with blood and the reagents typically used in test strips.

In some embodiments, the beads may not be colored. However, it is thought that the beads benefit from being colored in a color as similar to red blood cells as possible. This is because many of the detection techniques utilized in vertical flow test strips include detection of absorption or reflectivity using an optical meter. Vertical flow test strips, although composed of multiple layers, may be, in many configurations, very thin. Therefore, although the red blood cells or RFPs may not be directly on the measured reaction layer, their presence may still affect the optical properties of the reaction layer. The purpose of including the RFPs is to mimic the characteristics of red blood cells in the calibration samples from which they have been removed. Therefore, although the majority of red blood cells, and in the case of this modification of adding RFPs to serum, are filtered before reaching the reaction layer, the color provided by red blood cells or in this case RFPs may affect the measured absorption or reflectivity of the sample layer.

To expand on this idea, hematocrit bias (or red blood cell bias) may be an issue for vertical flow tests strips, not only in relation to the viscosity and kinetics of samples, but also to the absorption, reflectivity, or other color change that is measured. Therefore, in another embodiment, additional calibration tables and/or algorithms for meters can be created by providing different levels of RFPs to various samples; e.g., a meter may have algorithms based on calibration serum or plasma modified to mimic various hematocrit levels using RFPs. Additionally, such meters may not only be calibrated for measuring different levels of analytes, but also for measuring those analytes at different hematocrit levels. This especially is applicable to meters that may include a system for measuring hematocrit levels, such as those including an electrochemical technique for detecting hematocrit. In some alternatives, such an electrochemical method may be combined with glucose detection.

Therefore, various embodiments of serum or plasma for use in calibration of vertical flow test strips may include particles mimicking red blood cells. In many embodiments, these may be RFPs. Although red blood cells must be filtered in the vertical flow test strip system, the filtering and compensation is so integral to a vertical flow test strip that, without the red blood cells or a particle imitating them, the system fails to function properly.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof. Note that, although particular embodiments are shown, features of each attachment may be interchanged between embodiments.

What is claimed as new and desired to be protected by Letters Patent of the United States is:

1. A calibration sample comprising:
serum/plasma having a known level of a first analyte; and
a plurality of particles that mimic the characteristics of red blood cells.

2. The calibration sample of claim 1, wherein the plurality of particles are of red florescent particles.

3. The calibration sample of claim 2, wherein the red florescent particles are polystyrene.

4. The calibration sample of claim 2, wherein the red florescent particles are selected from the group consisting of other polymeric material and silica based particles.

5. The calibration sample of claim 3, wherein an amount of red florescent particles added to the serum/plasma is 20% of the plasma volume.

6. The calibration sample of claim 3, wherein the amount of red florescent particles added to the serum/plasma is between 5% and 50% of the plasma volume.

7. The calibration sample of claim 1, wherein the plurality of particles are non-dyed particles.

8. The calibration sample of claim 7, wherein an amount of non-dyed particles added to the serum/plasma is 20% of the plasma volume.

9. The calibration sample of claim 7, wherein the amount of non-dyed particles added to the serum/plasma is between 5% and 50% of the plasma volume.

10. The calibration sample of claim 1, wherein the first analyte is one of a plurality of analytes, the plurality of analytes consisting of HDL, LDL, triglycerides, creatinine, ketone, total cholesterol, and glucose.

11. A method of improving a calibration sample for use with a vertical flow test strip, comprising:
providing serum/plasma having a known level of a first analyte; and
adding a plurality of particles that mimic the characteristics of red blood cells.

12. The method of claim 11, wherein the plurality of particles are of red florescent particles.

13. The method of claim 12, wherein an amount of red florescent particles added to the serum/plasma is 20% of the plasma volume.

14. The method of claim 12, wherein the amount of red florescent particles added to the serum/plasma is between 5% and 50% of the plasma volume.

15. A method of calibrating a meter, the meter utilizing a vertical flow test strip, the method comprising:
providing serum/plasma having a known level of a first analyte; and
adjusting the viscosity of the serum/plasma by adding a plurality of particles that mimic the characteristics of red blood cells.

16. A method of adjusting calibration results of a vertical flow test strip having a limited dynamic range, calibrated with a calibration sample, the calibration sample including serum/plasma having a known level of a first analyte, the method comprising:
adding a plurality of particles that mimic the characteristics of red blood cells.

17. The method of claim 16, wherein the plurality of particles are of red florescent particles.

18. The method of claim 17, wherein an amount of red florescent particles added to the serum/plasma is 20% of the plasma volume.

19. The method of claim 17, wherein the amount of red florescent particles added to the serum/plasma is between 5% and 50% of the plasma volume.

20. The method of claim 16, wherein the first analyte is HDL.

21. The method of claim 20, wherein the calibration results are adjusted down to be within the upper limit of the limited dynamic range.
